SEQUENCE LISTING (6 sequences)

SEQ ID NO: 1 - protein, 520 aa, Bos taurus
MAQQRQHQRLPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 2 - DNA, 1923 bp, Bos taurus
  source 1..1923: mol_type = other DNA
ctcttctggagataccacttgatggcacagcagaggcagcatcagcgtcttcccatggatccgctgtgcacagcctcctcaggccctcggaagaagagacccaggcaggtgggtgcctcaatggcctcccctcctcatgacatcaagtttcaaaatttggtcctcttcattttggagaagaaaatgggaaccacccgcagaaacttcctcatggagctggctcgaaggaaaggtttcagggttgaaaatgagctcagtgattctgtcacccacattgtagcagaaaacaactctggttcagaggttctcgagtggcttcaggtacagaacataagagccagctcgcagctagaactccttgatgtctcctggctgatcgaaagtatgggagcaggaaaaccagtggagattacaggaaaacaccagcttgttgtgagaacagactattcagctaccccaaacccaggcttccagaagactccaccacttgctgtaaaaaagatctcccagtacgcgtgtcaaagaaaaaccactttgaacaactataaccacatattcacggatgcctttgagatactggctgaaaattctgagtttaaagaaaatgaagtctcttatgtgacatttatgagagcagcttctgtacttaaatctctgccattcacaatcatcagtatgaaggatacagaaggaattccctgcctgggggacaaggtgaagtgtatcatagaggaaattattgaagatggagaaagttctgaagttaaagctgtgttaaatgatgaacgatatcagtccttcaaactctttacttctgtttttggagtgggactgaagacatctgagaaatggttcaggatggggttcagatctctgagtaaaataatgtcagacaaaaccctgaaattcacaaaaatgcagaaagcaggatttctctattatgaagaccttgtcagctgcgtgaccagggccgaagcagaggcggttggcgtgctggttaaagaggctgtgtgggcatttctgccggatgcctttgtcaccatgacaggaggattccgcaggggtaagaagattgggcatgatgtagattttttaattaccagcccaggatcagcagaggatgaagagcaacttttgcctaaagtgataaacttatgggaaaaaaagggattacttttatattatgaccttgtggagtcaacatttgaaaagttcaagttgccaagcaggcaggtggatactttagatcattttcaaaaatgctttctgattttaaaattgcaccatcagagagtagacagtagcaagtccaaccagcaggaaggaaagacctggaaggccatccgtgtggacctggttatgtgcccctacgagaaccgtgcctttgccctgctaggctggactggctcccggcagtttgagagagacatccggcgctatgccacacacgagcggaagatgatgctggataaccacgctttatatgacaagaccaagagggtatttctcaaagcggaaagtgaagaagaaatctttgcacatctgggattggactacattgaaccatgggaaagaaatgcttaggagaaagctgtcaacttttttcttttctgttctttttttcaggttagacaaattatgcttcatattataatgaaagatgccttagtcaagtttgggattctttacattttaccaagatgtagattgcttctagaaataagtagttttggaaacgtgatcaggcaccccctgggttatgctctggcaagccatttgcaggactgatgtgtagaactcgcaatgcattttccatagaaacagtgttggaattggtggctcatttccagggaagttcatcaaagcccactttgcccacagtgtagctgaaatactgtatacttgccaataaaaataggaaac

SEQ ID NO: 3 - protein, 465 aa, Escherichia coli
MFTRVANFCRKVLSREESEAEQAVARPQVTVIPREQHAISRKDISENALKVMYRLNKAGYEAWLVGGGVRDLLLGKKPKDFDVTTNATPEQVRKLFRNCRLVGRRFRLAHVMFGPEIIEVATFRGHHEGNVSDRTTSQRGQNGMLLRDNIFGSIEEDAQRRDFTINSLYYSVADFTVRDYVGGMKDLKDGVIRLIGNPETRYREDPVRMLRAVRFAAKLGMRISPETAEPIPRLATLLNDIPPARLFEESLKLLQAGYGYETYKLLCEYHLFQPLFPTITRYFTENGDSPMERIIEQVLKNTDTRIHNDMRVNPAFLFAAMFWYPLLETAQKIAQESGLTYHDAFALAMNDVLDEACRSLAIPKRLTTLTRDIWQLQLRMSRRQGKRAWKLLEHPKFRAAYDLLALRAEVERNAELQRLVKWWGEFQVSAPPDQKGMLNELDEEPSPRRRTRRPRKRAPRREGTA

SEQ ID NO: 4 - DNA, 1398 bp, Escherichia coli
  source 1..1398: mol_type = other DNA
atttttacccgagtcgctaatttttgccgcaaggtgctaagccgcgaggaaagcgaggctgaacaggcagtcgcccgtccacaggtgacggtgatcccgcgtgagcagcatgctatttcccgcaaagatatcagtgaaaatgccctgaaggtaatgtacaggctcaataaagcgggatacgaagcctggctggttggcggcggcgtgcgcgacctgttacttggcaaaaagccgaaagattttgacgtaaccactaacgccacgcctgagcaggtgcgcaaactgttccgtaactgccgcctggtgggtcgccgtttccgtctggctcatgtaatgtttggcccggagattatcgaagttgcgaccttccgtggacaccacgaaggtaacgtcagcgaccgcacgacctcccaacgcgggcaaaacggcatgttgctgcgcgacaacattttcggctccatcgaagaagacgcccagcgccgcgatttcactatcaacagcctgtattacagcgtagcggattttaccgtccgtgattacgttggcggcatgaaggatctgaaggacggcgttatccgtctgattggtaacccggaaacgcgctaccgtgaagatccggtacgtatgctgcgcgcggtacgttttgccgccaaattgggtatgcgcatcagcccggaaaccgcagaaccgatccctcgcctcgctaccctgctgaacgatatcccaccggcacgcctgtttgaagaatcgcttaaactgctacaagcgggctacggttacgaaacctataagctgttgtgtgaatatcatctgttccagccgctgttcccgaccattacccgctacttcacggaaaatggcgacagcccgatggagcggatcattgaacaggtgctgaagaataccgatacgcgtatccataacgatatgcgcgtgaacccggcgttcctgtttgccgccatgttctggtacccactgctggagacggcacagaagatcgcccaggaaagcggcctgacctatcacgacgctttcgcgctggcgatgaacgacgtgctggacgaagcctgccgttcactggcaatcccgaaacgtctgacgacattaacccgcgatatctggcagttgcagttgcgtatgtcccgtcgtcagggtaaacgcgcatggaaactgctggagcatcctaagttccgtgcggcttatgacctgttggccttgcgagctgaagttgagcgtaacgctgaactgcagcgtctggtgaaatggtggggtgagttccaggtttccgcgccaccagaccaaaaagggatgctcaacgagctggatgaagaaccgtcaccgcgtcgtcgtactcgtcgtccacgcaaacgcgcaccacgtcgtgagggtaccgcatga

SEQ ID NO: 5 - protein, 349 aa, Escherichia coli
GSHMSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEA

SEQ ID NO: 6 - DNA, 1047 bp, Escherichia coli
  source 1..1047: mol_type = other DNA
ggcagccatatgagctatcagaaagtgccgaacagccataaagaatttaccaaattttgctatgaagtgtataacgaaattaaaattagcgataaagaatttaaagaaaaacgcgcggcgctggataccctgcgcctgtgcctgaaacgcattagcccggatgcggaactggtggcgtttggcagcctggaaagcggcctggcgctgaaaaacagcgatatggatctgtgcgtgctgatggatagccgcgtgcagagcgataccattgcgctgcagttttatgaagaactgattgcggaaggctttgaaggcaaatttctgcagcgcgcgcgcattccgattattaaactgaccagcgataccaaaaacggctttggcgcgagctttcagtgcgatattggctttaacaaccgcctggcgattcataacaccctgctgctgagcagctataccaaactggatgcgcgcctgaaaccgatggtgctgctggtgaaacattgggcgaaacgcaaacagattaacagcccgtattttggcaccctgagcagctatggctatgtgctgatggtgctgtattatctgattcatgtgattaaaccgccggtgtttccgaacctgctgctgagcccgctgaaacaggaaaaaattgtggatggctttgatgtgggctttgatgataaactggaagatattccgccgagccagaactatagcagcctgggcagcctgctgcatggcttttttcgcttttatgcgtataaatttgaaccgcgcgaaaaagtggtgacctttcgccgcccggatggctatctgaccaaacaggaaaaaggctggaccagcgcgaccgaacataccggcagcgcggatcagattattaaagatcgctatattctggcgattgaagatccgtttgaaattagccataacgtgggccgcaccgtgagcagcagcggcctgtatcgcattcgcggcgaatttatggcggcgagccgcctgctgaacagccgcagctatccgattccgtatgatagcctgtttgaagaagcg

(19) United States
(12) Patent Application Publication
(10) Pub. No.: US 2025/0197921 A1
(43) Pub. Date: Jun. 19, 2025

(54) METHODS AND APPARATUS FOR SYNTHESIZING NUCLEIC ACIDS

(71) Applicant: Molecular Assemblies, Inc., San Diego, CA (US)

(72) Inventors: J. William Efcavitch, San Carlos, CA (US); Suhaib Siddiqi, Burlington, MA (US)

(21) Appl. No.: 18/891,831

(22) Filed: Sep. 20, 2024

Related U.S. Application Data
(63) Continuation of application No. 17/373,314, filed on Jul. 12, 2021, now abandoned.
Application No. 17/373,314 is a continuation of application No. 16/026,662, filed on Jul. 3, 2018, now abandoned.
Application No. 16/026,662 is a continuation of application No. 14/444,440, filed on Jul. 28, 2014, now Pat. No. 10,041,110.
Application No. 14/444,440 is a continuation of application No. 14/056,687, filed on Oct. 17, 2013, now Pat. No. 8,808,989.
(60) Provisional application No. 61/891,162, filed on Oct. 15, 2013.
(60) Provisional application No. 61/807,327, filed on Apr. 2, 2013.

(51) Int. Cl. C12Q 1/6844; C12N 9/12; C12P 19/34; C12Q 1/6806

(52) U.S. Cl. CPC C12Q 1/6844; C12N 9/1241; C12N 9/1264; C12P 19/34; C12Q 1/6806; C12Y 207/07019; C12Y 207/07031; Y02P 20/582

(57) ABSTRACT

The invention provides improved methods for synthesizing polynucleotides, such as DNA and RNA, using enzymes and specially designed nucleotide analogs. Using the methods of the invention, specific sequences of polynucleotides can be synthesized de nova, base by base, in an aqueous environment, without the use of a nucleic acid template. Because the nucleotide analogs have an unmodified 3′ OH, i.e., as found in “natural” deoxyribose and ribose molecules, the analogs result in natural polynucleotides suitable for incorporation into biological systems.

### RELATED APPLICATIONS

This Application is a continuation of U.S. patent application Ser. No. 17/373,314, filed Jul. 12, 2021, which is a continuation of U.S. patent application Ser. No. 16/026,662, filed Jul. 3, 2018, which is a continuation of U.S. patent application Ser. No. 14/444,440, filed Jul. 28, 2014, which is a continuation of U.S. patent application Ser. No. 14/056,687, filed Oct. 17, 2013, which claims priority to U.S. Provisional Application Nos. 61/807,327, filed Apr. 2, 2013, and 61/891,162, filed Oct. 15, 2013, all of which are incorporated by reference in their entireties.

### SEQUENCE LISTING

This application contains a Sequence Listing which has been submitted in XML format and is hereby incorporated by reference in its entirety. The Sequence Listing was created on Sep. 19, 2024, is named MOLA-001_13US.xml and is 11,854 bytes in size.

### FIELD OF THE INVENTION

The invention relates to methods and apparatus for synthesizing polynucleotides (de nova) with a desired sequence and without the need for a template. As such, the invention provides the capacity to make libraries of polynucleotides of varying sequence and varying length for research, genetic engineering, and gene therapy.

### BACKGROUND

Genetic engineering requires tools for determining the content of genetic material as well as tools for constructing desired genetic materials. The tools for determining the content of genetic material have made it possible to sequence an entire human genome in about one day for under $1,000. (See Life Technologies, Press Release: Benchtop Ion Proton™ Sequencer, Jan. 10, 2012). In contrast, the tools for constructing desired genetic materials, e.g., de nova DNA synthesis, have not improved at the same pace. As a point of reference, over the past 25 years, the cost (per base) of de nova small nucleic acid synthesis has dropped 10-fold, while the cost (per base) of nucleic acid sequencing has dropped over 10,000,000-fold. The lack of progress in DNA synthesis now limits the pace of translational genomics, i.e., whereby the role of individual sequence variations are determined and used to develop therapeutic treatments.

Currently, most de nova nucleic acid sequences are synthesized using solid phase phosphoramidite-techniques developed more than 30 years ago. The technique involves the sequential de-protection and synthesis of sequences built from phosphoramidite reagents corresponding to natural (or non-natural) nucleic acid bases. Phosphoramidite nucleic acid synthesis is length-limited, however, in that nucleic acids greater than 200 base pairs (bp) in length experience high rates of breakage and side reactions. Additionally, phosphoramidite synthesis produces toxic by-products, and the disposal of this waste limits the availability of nucleic acid synthesizers and increases the costs of contract oligo production. (It is estimated that the annual demand for oligonucleotide synthesis is responsible for greater than 300,000 gallons of hazardous chemical waste, including acetonitrile, trichloroacetic acid, toluene, tetrahydrofuran, and pyridine. See LeProust et al., Nucleic Acids Res., vol. 38(8), p.2522-2540, (2010), incorporated by reference herein in its entirety). Thus, there is a need for more efficient and cost-effective methods for oligonucleotide synthesis.

### SUMMARY

The invention provides improved methods for nucleic acid synthesis. Methods of the invention provide faster and longer de nova synthesis of polynucleotides. As such, the invention dramatically reduces the overall cost of synthesizing custom nucleic acids. Methods of the invention are directed to template-independent synthesis of polynucleotides by using a nucleotidyl transferase enzyme to incorporate nucleotide analogs having an unmodified 3′ hydroxyl and a cleavable terminating group. Because of the terminating group, synthesis pauses with the addition of each new base, whereupon the terminating group is cleaved, leaving a polynucleotide that is essentially identical to a naturally occurring nucleotide (i.e., is recognized by the enzyme as a substrate for further nucleotide incorporation).

The invention additionally includes an apparatus that utilizes methods of the invention for the production of custom polynucleotides. An apparatus of the invention includes one or more bioreactors providing aqueous conditions and a plurality of sources of nucleotide analogs. The bioreactor may be e.g., a reservoir, a flow cell, or a multi-well plate. Starting from a solid support, the polynucleotides are grown in the reactor by adding successive nucleotides via the natural activity of a nucleotidyl transferase, e.g., a terminal deoxynucleotidyl transferase (TdT) or any other enzyme which elongates DNA or RNA strands without template direction. Upon cleavage of the terminating group, a natural polynucleotide is exposed on the solid support.

Once the sequence is complete, the support is cleaved away, leaving a polynucleotide essentially equivalent to that found in nature. In some embodiments, the apparatus is designed to recycle nucleotide analog solutions by recovering the solutions after nucleotide addition and reusing solutions for subsequence nucleotide addition. Thus, less waste is produced, and the overall cost per base is reduced as compared to state-of-the-art methods.

Other aspects of the invention are apparent to the skilled artisan upon consideration of the following figures and detailed description.

### BRIEF DESCRIPTION OF THE DRAWINGS

FIG. 1A shows a genus of deoxycytidine triphosphate (dCTP) analogs having a cleavable terminator linked at the N-4 position;

FIG. 1B shows cleavage of the cleavable terminator from a dCTP analog of FIG. 1A to achieve a “natural” dCTP and a cyclic leaving molecule;

FIG. 2A shows a genus of deoxyadenosine triphosphate (dATP) analogs having a cleavable terminator linked at the N-6 position;

FIG. 2B shows cleavage of the cleavable terminator from a dATP analog of FIG. 2A to achieve a “natural” dATP and a cyclic leaving molecule;

FIG. 3A shows a genus of deoxyguanosine triphosphate (dGTP) analogs having a cleavable terminator linked at the N-2 position;

FIG. 3B shows cleavage of the cleavable terminator from a dGTP analog of FIG. 3A to achieve a “natural” dGTP and a cyclic leaving molecule;

FIG. 4A shows a genus of deoxythymidine triphosphate (dTTP) analogs having a cleavable terminator linked at the N-3 position;

FIG. 4B shows cleavage of the cleavable terminator from a dTTP analog of FIG. 4A to achieve a “natural” dTTP and a cyclic leaving molecule;

FIG. 5A shows a genus of deoxyuridine triphosphate (dUTP) analogs having a cleavable terminator linked at the N-3 position;

FIG. 5B shows cleavage of the cleavable terminator from a dUTP analog of FIG. 5A to achieve a dUTP and a cyclic leaving molecule;

FIG. 6 shows an exemplary deoxycytidine triphosphate (dCTP) analog having a Staudinger linker connecting a blocking Asp-Asp molecule to the N-4 position of the deoxycytidine and subsequent cleavage of the Staudinger linker under aqueous conditions to achieve a dCTP and a leaving group; FIG. 7A shows a genus of cytidine triphosphate (rCTP) analogs having a cleavable terminator linked at the N-4 position;

FIG. 7B shows cleavage of the cleavable terminator from a rCTP analog of FIG. 7A to achieve a “natural” rCTP and a cyclic leaving molecule;

FIG. 8A shows a genus of adenosine triphosphate (rATP) analogs having a cleavable terminator linked at the N-6 position;

FIG. 8B shows cleavage of the cleavable terminator from an rATP analog of FIG. 8A to achieve a “natural” rATP and a cyclic leaving molecule;

FIG. 9A shows n genus of guanosine triphosphate (rGTP) analogs having a cleavable terminator linked at the N-2 position;

FIG. 9B shows cleavage of the cleavable terminator from a rGTP analog of FIG. 9A to achieve a “natural” rGTP and a cyclic leaving molecule;

FIG. 10A shows a genus of thymidine triphosphate (rTTP) analogs having a cleavable terminator linked at the N-3 position;

FIG. 10B shows cleavage of the cleavable terminator from a rTTP analog of FIG. 10A to achieve a “natural” rTTP and a cyclic leaving molecule;

FIG. 11A shows a genus of uridine triphosphate (rUTP) analogs having a cleavable terminator linked at the N-3 position;

FIG. 11B shows cleavage of the cleavable terminator from a rUTP analog of FIG. 11A to achieve a rUTP and a cyclic leaving molecule;

FIG. 12 shows an exemplary cytidine triphosphate (rCTP) analog having a Staudinger linker connecting a blocking Asp-Asp molecule to the N-4 position of the cytidine and subsequent cleavage of the Staudinger linker under aqueous conditions to achieve a rCTP and a leaving group;

FIG. 13 shows an exemplary terminal deoxynucleotidyl transferase (TdT) mediated polynucleotide synthetic cycle, including: (a) incorporation of a nucleotide triphosphate analog comprising cleavable terminator, dN*TP-OH, and (b) removal of the terminating blocking group (indicated by*), thus enabling the next dN*TP-OH to be incorporated, wherein N=A, G, C, or T.

### DETAILED DESCRIPTION

The invention provides improved methods for synthesizing polynucleotides, such as DNA and RNA, using enzymes and nucleic acid analogs. Using the disclosed methods, specific sequences of polynucleotides can be synthesized de nova, base by base, in an aqueous environment, without the use of a nucleic acid template. Additionally, because the nucleotide analogs have an unmodified 3′ hydroxyls, i.e., as found in “natural” deoxyribose and ribose molecules, the analogs result in “natural” nucleotides when a cleavable blocking group is removed from the base. Other nucleotide analogs can also be used which, for example, include self-eliminating linkers, or nucleotides with modified phosphate groups. In most instances, the blocking group is designed to not leave behind substantial additional molecules, i.e., designed to leave behind “scarless” nucleotides that are recognized as “natural” nucleotides by the enzyme. Thus, at the conclusion of the synthesis, upon removal of the last blocking group, the synthesized polynucleotide is chemically and structurally equivalent to the naturally occurring polynucleotide with the same sequence. The synthetic polynucleotide can, thus, be incorporated into living systems without concern that the synthesized polynucleotide will interfere with biochemical pathways or metabolism.

The process and analogs of the current invention can be used for the non-templated enzymatic synthesis of useful oligo-and oligodeoxynucleotides especially of long oligonucleotides (<5000 nt). Products can be single strand or partially double strand depending upon the initiator used. The synthesis of long oligonucleotides requires high efficiency incorporation and high efficiency of reversible terminator removal. The initiator bound to the solid support consists of a short, single strand DNA sequence that is either a short piece of the user defined sequence or a universal initiator from which the user defined single strand product is removed.

In one aspect, the disclosed methods employ commercially available nucleotidyl transferase enzymes, such as terminal deoxynucleotidyl transferase (TdT), to synthesize polynucleotides from nucleotide analogs in a step-by-step fashion. The nucleotide analogs are of the form:
NTP-linker-inhibitor
   wherein NTP is a nucleotide triphosphate (i.e., a dNTP or an rNTP), the linker is a cleavable linker between the pyridine or pyrimidine of the base, and the inhibitor is a group that prevents the enzyme from incorporating subsequent nucleotides. At each step, a new nucleotide analog is incorporated into the growing polynucleotide chain, whereupon the enzyme is blocked from adding an additional nucleotide by the inhibitor group. Once the enzyme has stopped, the excess nucleotide analogs can be removed from the growing chain, the inhibitor can be cleaved from the NTP, and new nucleotide analogs can be introduced in order to add the next nucleotide to the chain. By repeating the steps sequentially, it is possible to quickly construct nucleotide sequences of a desired length and sequence. Advantages of using nucleotidyl transferases for polynucleotide synthesis include: 1) 3′-extension activity using single strand (ss) initiating primers in a template-independent polymerization, 2) the ability to extend primers in a highly efficient manner resulting in the addition of thousands of nucleotides, and 3) the acceptance of a wide variety of modified and substituted NTPs as efficient substrates. In addition, the invention can make use of an initiator sequence that is a substrate for nucleotidyl transferase. The initiator is attached to a solid support and serves as a binding site for the enzyme. The initiator is preferably a universal initiator for the enzyme, such as a homopolymer sequence and is recyclable on the solid support, the formed oligonucleotide being cleavable from the initiator.

Methods of the invention are well-suited to a variety of applications that currently use synthetic nucleic acids, e.g., phosphoramidite-synthesized DNA oligos. For example, polynucleotides synthesized with the methods of the invention can be used as primers for nucleic acid amplification, hybridization probes for detection of specific markers, and for incorporation into plasmids for genetic engineering. However, because the disclosed methods produce longer synthetic strings of nucleotides, at a faster rate, and in an aqueous environment, the disclosed methods also lend themselves to high-throughput applications, such as screening for expression of genetic variation in cellular assays, as well as synthetic biology. Furthermore, the methods of the invention will provide the functionality needed for next-generation applications, such as using DNA as synthetic read/write memory, or creating macroscopic materials synthesized completely (or partially) from DNA.

The invention and systems described herein provide for synthesis of polynucleotides, including deoxyribonucleic acids (DNA) and ribonucleic acids (RNA). While synthetic pathways for “natural” nucleotides, such as DNA and RNA, are described in the context of the common nucleic acid bases, e.g., adenine (A), guanine (G), cytosine (C), thymine (T), and uracil (U), it is to be understood that the methods of the invention can be applied to so-called “non-natural” nucleotides, including nucleotides incorporating universal bases such as 3-nitropyrrole 2′-deoxynucloside and 5-nitroindole 2′-deoxynucleoside, alpha phosphorothiolate, phosphorothioate nucleotide triphosphates, or purine or pyrimidine conjugates that have other desirable properties, such as fluorescence. Other examples of purine and pyrimidine bases include pyrazolo [3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo [3,4-d]pyrimidine, imidazo [1,5-a]1,3,5 triazinones, 9-deazapurines, imidazo [4,5-d]pyrazines, thiazolo [4,5-d]pyrimidines, pyrazin-2-ones, 1,2,4-triazine, pyridazine; and 1,3,5 triazine. In some instances, it may be useful to produce nucleotide sequences having unreactive, but approximately equivalent bases, i.e., bases that do not react with other proteins, i.e., transcriptases, thus allowing the influence of sequence information to be decoupled from the structural effects of the bases.

### Analogs

The invention provides nucleotide analogs having the formula NTP-linker-inhibitor for synthesis of polynucleotides in an aqueous environment. With respect to the analogs of the form NTP-linker-inhibitor, NTP can be any nucleotide triphosphate, such as adenosine triphosphate (ATP), guanosine triphosphate (GTP), cytidine triphosphate (CTP), thymidine triphosphate (TTP), uridine triphosphate (UTP), nucleotide triphosphates, deoxyadenosine triphosphate (dATP), deoxyguanosine triphosphate (dGTP), deoxycytidine triphosphate (dCTP), deoxythymidine triphosphate (dTTP), or deoxyuridine triphosphate (dUTP).

The linker can be any molecular moiety that links the inhibitor to the NTP and can be cleaved, e.g., chemically cleaved, electrochemically cleaved, enzymatically cleaved, or photolytically cleaved. For example, the linkers can be cleaved by adjusting the pH of the surrounding environment. The linkers may also be cleaved by an enzyme that is activated at a given temperature, but inactivated at another temperature. In some embodiments, the linkers include disulfide bonds.

The linker can be attached, for example, at the N4 of cytosine, the N3 or 04 of thymine, the N2 or N3 of guanine, and the N6 of adenine, or the N3 or 04 of uracil because attachment at a carbon results in the presence of a residual scar after removal of the polymerase-inhibiting group. The linker is typically on the order of at least about 10 Angstroms long, e.g., at least about 20 Angstroms long, e.g., at least about 25 Angstroms long, thus allowing the inhibitor to be far enough from the pyridine or pyrimidine to allow the enzyme to bind the NTP to the polynucleotide chain via the attached sugar backbone. In some embodiments, the cleavable linkers are self-cyclizing in that they form a ring molecule that is particularly non-reactive toward the growing nucleotide chain.

The nucleotide analogs can include any moiety linked to the NTP that inhibits the coupling of subsequent nucleotides by the enzyme. The inhibitory group can be a charged group, such as a charged amino acid, or the inhibitory group can be a group that becomes charged depending upon the ambient conditions. In some embodiments, the inhibitor may include a moiety that is negatively charged or capable of becoming a negatively charged. In other embodiments, the inhibitor group is positively charged or capable of becoming positively charged. In some other embodiments, the inhibitor is an amino acid or an amino acid analog.

The inhibitor may be a peptide of 2 to 20 units of amino acids or analogs, a peptide of 2 to 10 units of amino acids or analogs, a peptide of 3 to 7 units of amino acids or analogs, a peptide of 3 to 5 units of amino acids or analogs. In some embodiments, the inhibitor includes a group selected from the group consisting of Glu, Asp, Arg, His, and Lys, and a combination thereof (e.g., Arg, Arg-Arg, Asp, Asp-Asp, Asp, Glu, Glu-Glu, Asp-Glu-Asp, Asp-Asp-Glu or AspAspAspAsp, etc.). Peptides or groups may be combinations of the same or different amino acids or analogs. The inhibitory group may also include a group that reacts with residues in the active site of the enzyme thus interfering with the coupling of subsequent nucleotides by the enzyme.

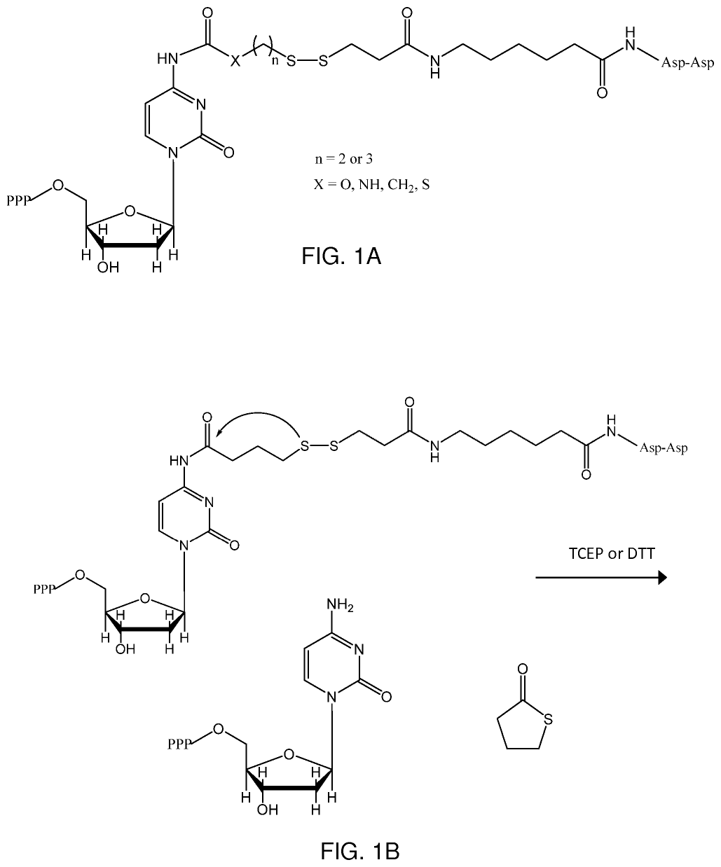

An example of a nucleotide analog of the type NTP-linker-inhibitor is shown in FIG. 1A. The analog in FIG. 1A includes an inhibitory (-Asp-Asp-) group linked to the N4 position of dCTP through a disulfide (—S—S—) bond while providing an unblocked, unmodified 3′-OH on the sugar ring. The linker is constructed such that all linker atoms (including the 2nd incorporation-inhibiting moiety) can be removed, thereby allowing the nascent DNA strand to revert to natural nucleotides. As shown in FIG. 1B, an aqueous reducing agent, such as tris (2-carboxyethyl) phosphine (TCEP) or dithiothreitol (DTT), can be used to cleave the —S—S-bond, resulting in the loss of the inhibitor function (deblocking). As shown in FIG. 1B, a self-cyclizing linker can be incorporated, resulting in a cyclic oxidized tetrahydrothiophene leaving group that is easily removed from the reagent solution at the conclusion of nucleotide synthesis.

An exemplary scheme for synthesizing a dCTP analog of FIG. 1A is shown below:

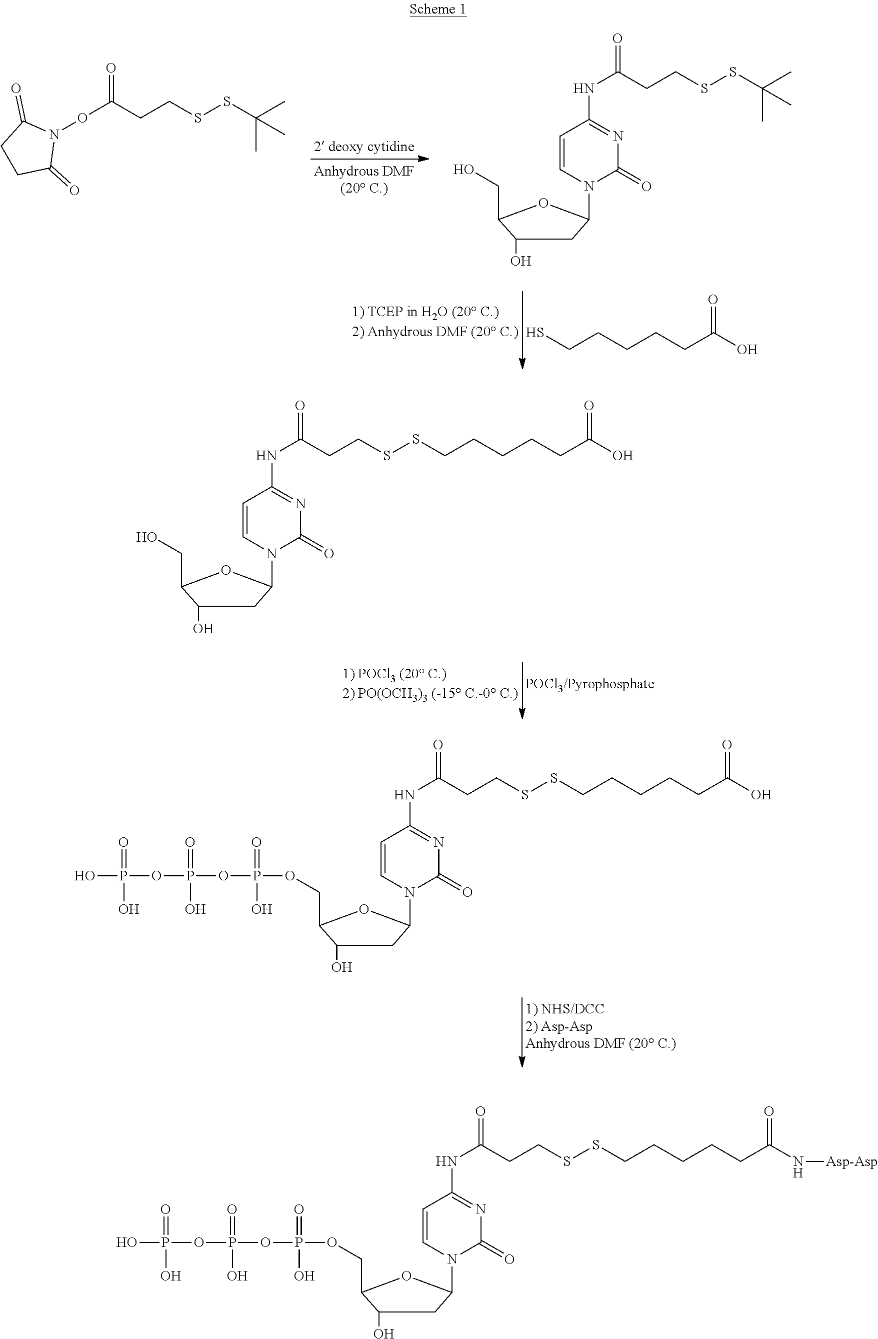

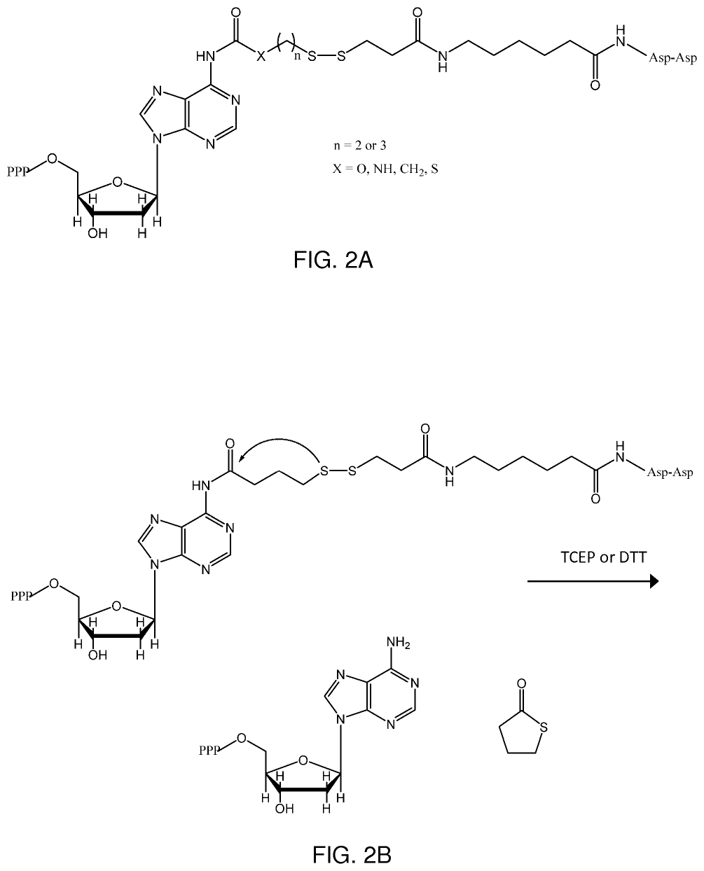

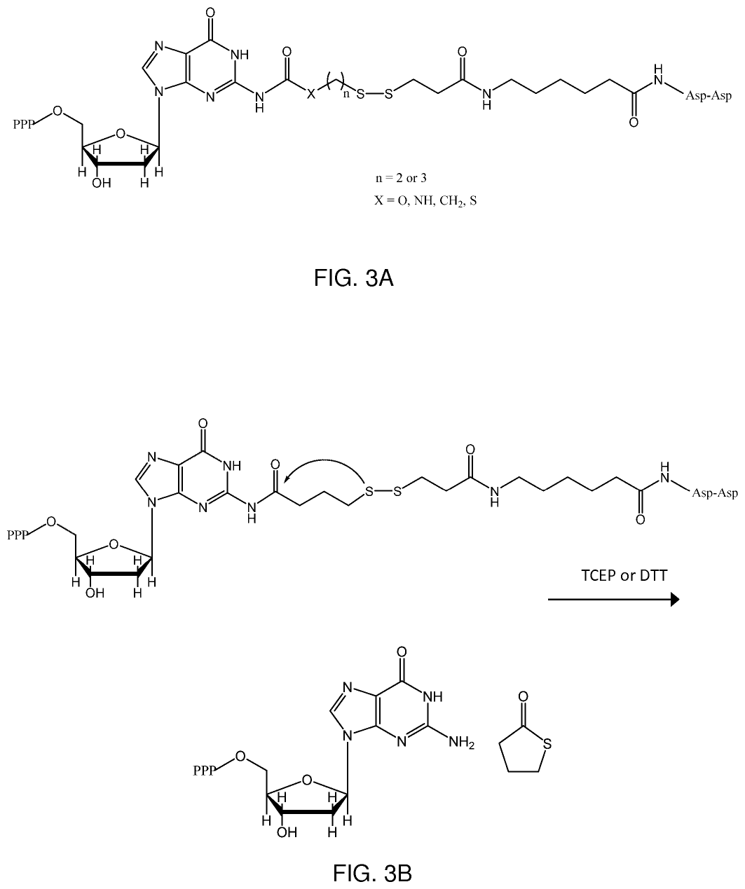

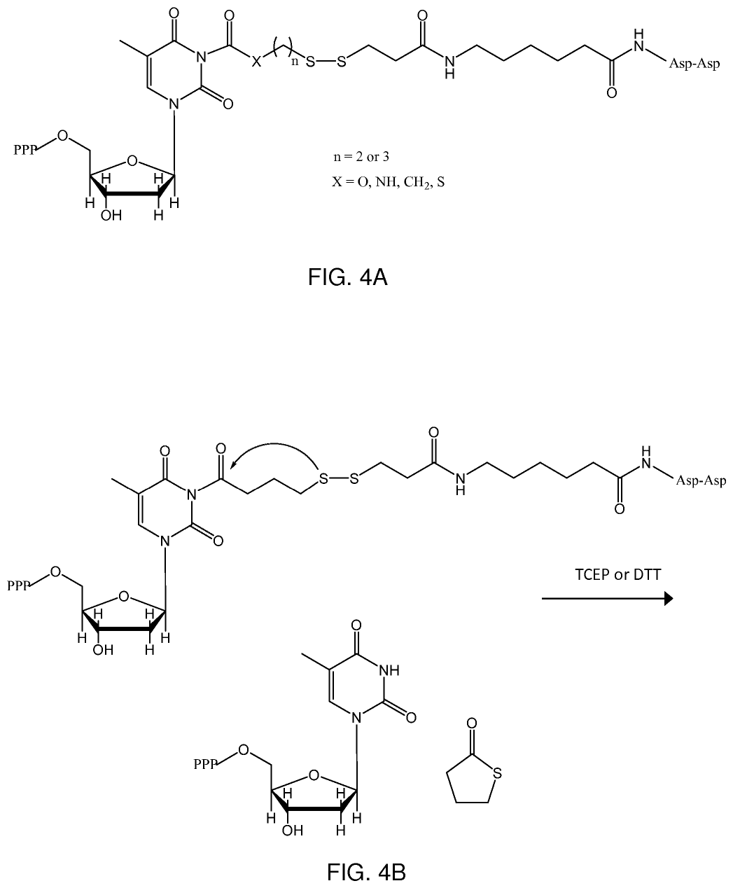

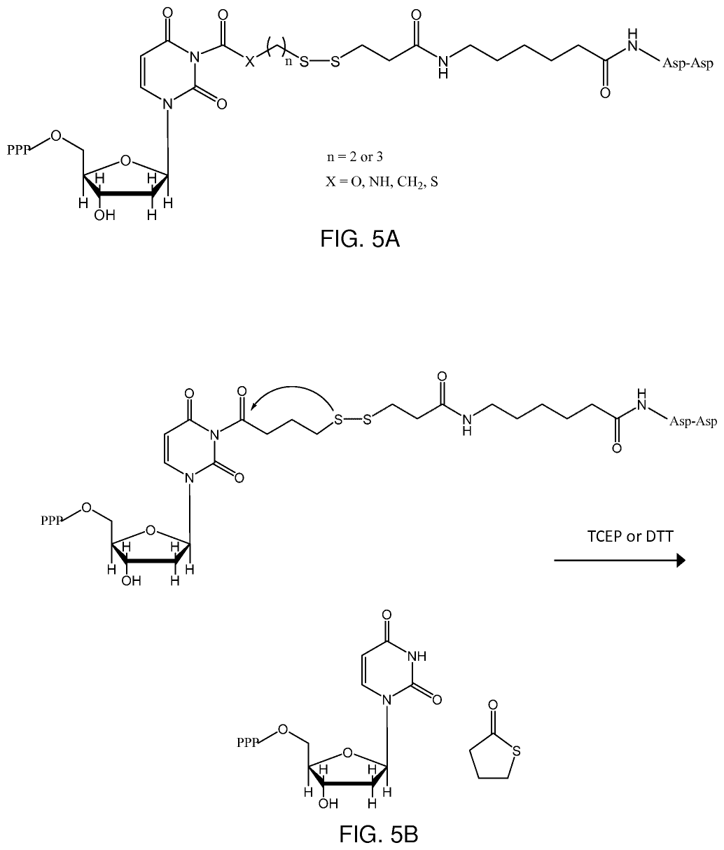

In a fashion analogous to FIG. 1, nucleotide analogs of the type NTP-linker-inhibitor can also be formed by attaching the linker-inhibitor moiety to the N6 of adenine (FIG. 2), the N2 of guanine (FIG. 3), the N3 of thymine (FIG. 4), or the N3 of uracil (FIG. 5), thereby providing analogs of the “naturally-occurring” dNTPs, as well as a deoxyuracil nucleotide (dUTP). While it is unlikely that there will be wide use of a dUTP, the synthesis is straightforward based upon the chemistry.

The invention is not limited to the linking chemistry of Scheme 1, however, as carbamate, amide, or other self-eliminating linkages could also be employed. For example, nucleotides can also be prepared with Staudinger linkers, as shown in Scheme 2.

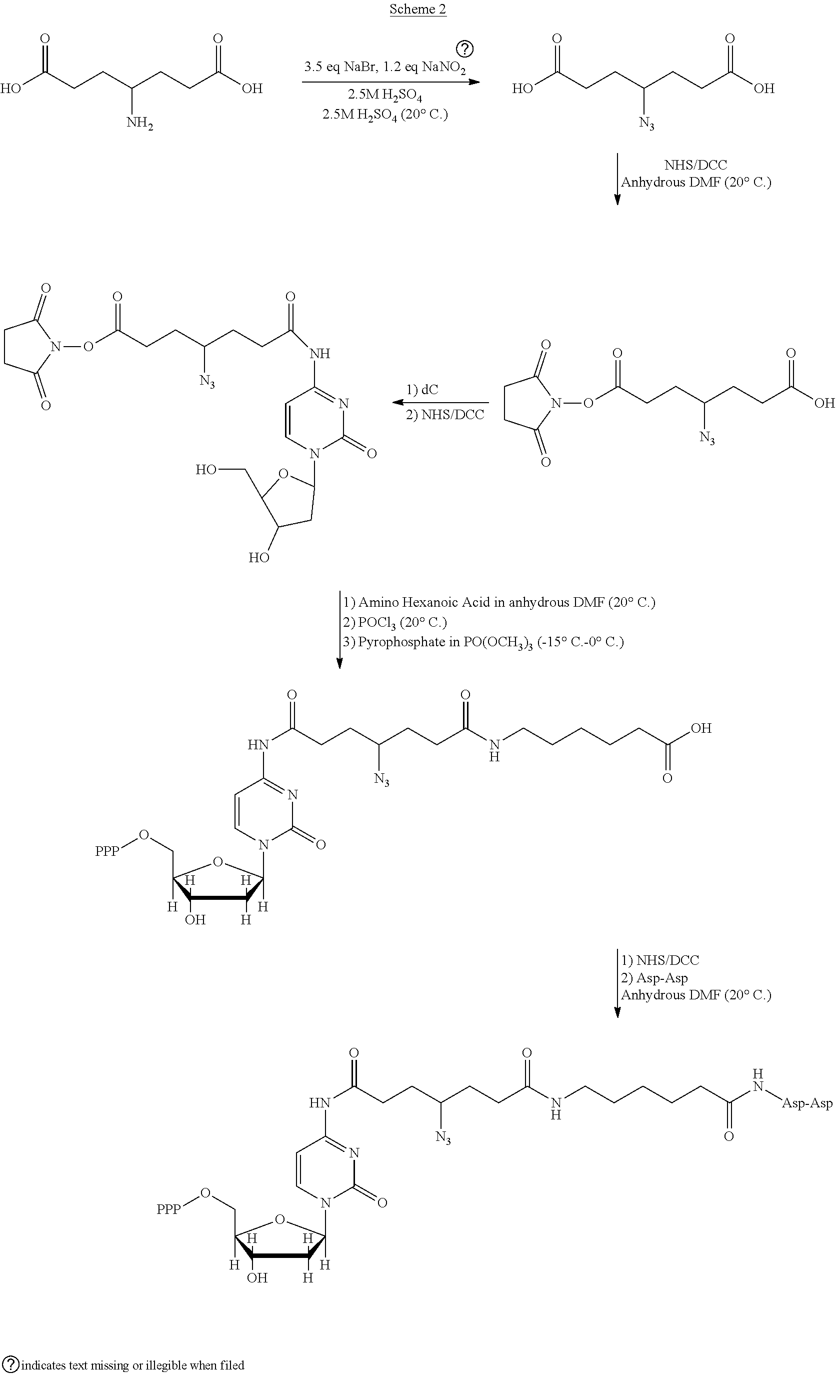

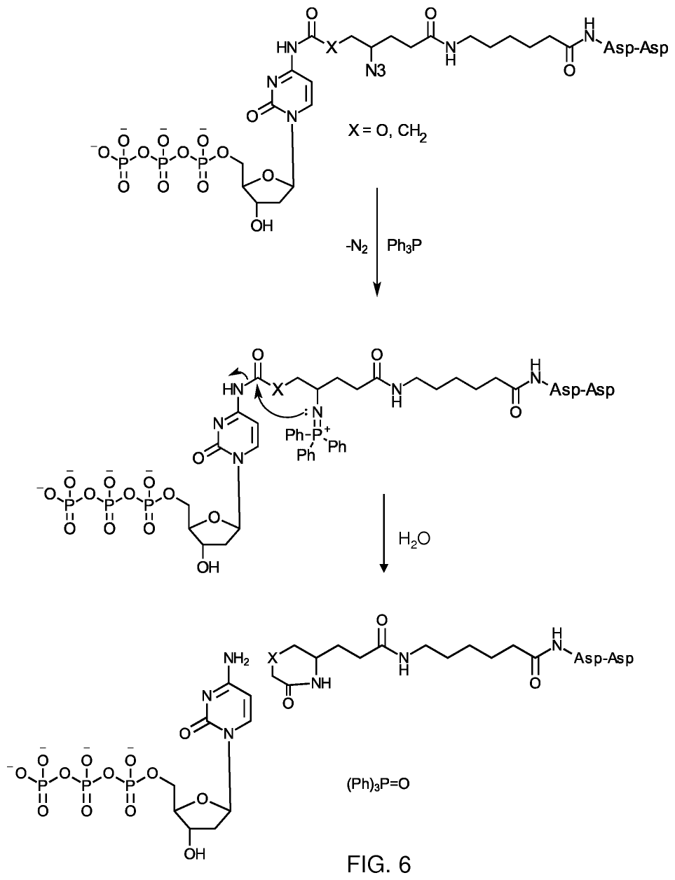

A deoxycytidine triphosphate (dCTP) analog created with a Staudinger linker (Scheme 2) to an Asp-Asp blocking group is shown in FIG. 6. As shown in FIG. 6, the Staudinger dCTP analog undergoes cleavage under aqueous conditions with the addition of azide and triphenylphosphine. The Staudinger analog shown in FIG. 6 is also suitable for nucleotide extension using nucleotidyl transferases, such as TdT, as described above and exemplified in FIGS. 1-5. While not shown explicitly in the FIGS., one of skill in the art can use Scheme 2 in conjunction with the suitable reactant to produce other nucleotide analogs having Staudinger linkers as needed for complete de nova nucleotide synthesis. In a fashion analogous to FIG. 6, nucleotide analogs of Scheme 2 can be formed by attaching the Staudinger moiety to the N6 of adenine, the N2 of guanine, the N3 of thymine, or the N3 of uracil, thereby providing analogs of the “naturally occurring” dNTPs, as well as a deoxyuracil nucleotide (dUTP).

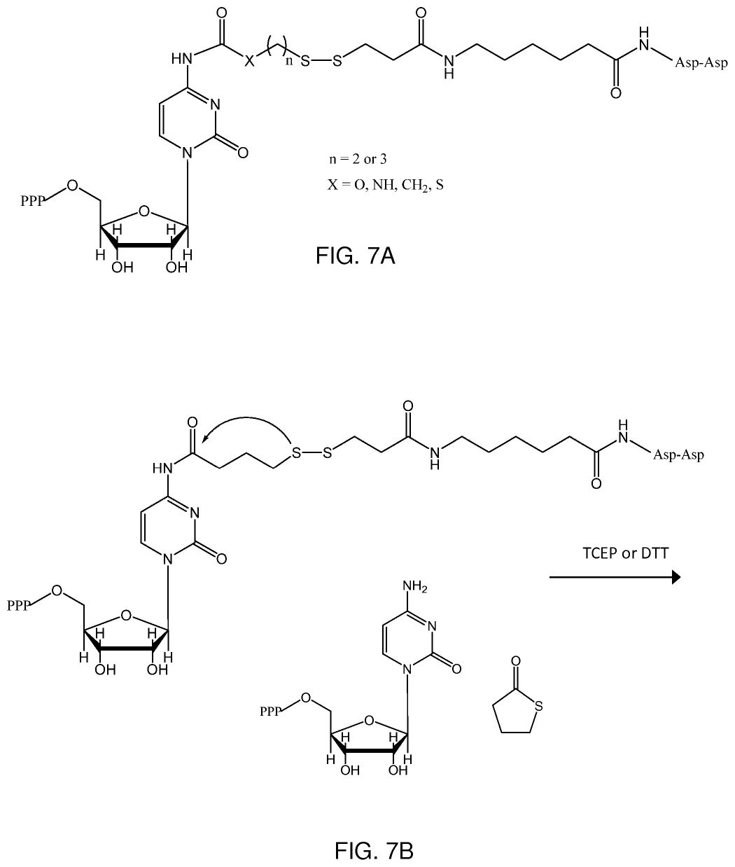

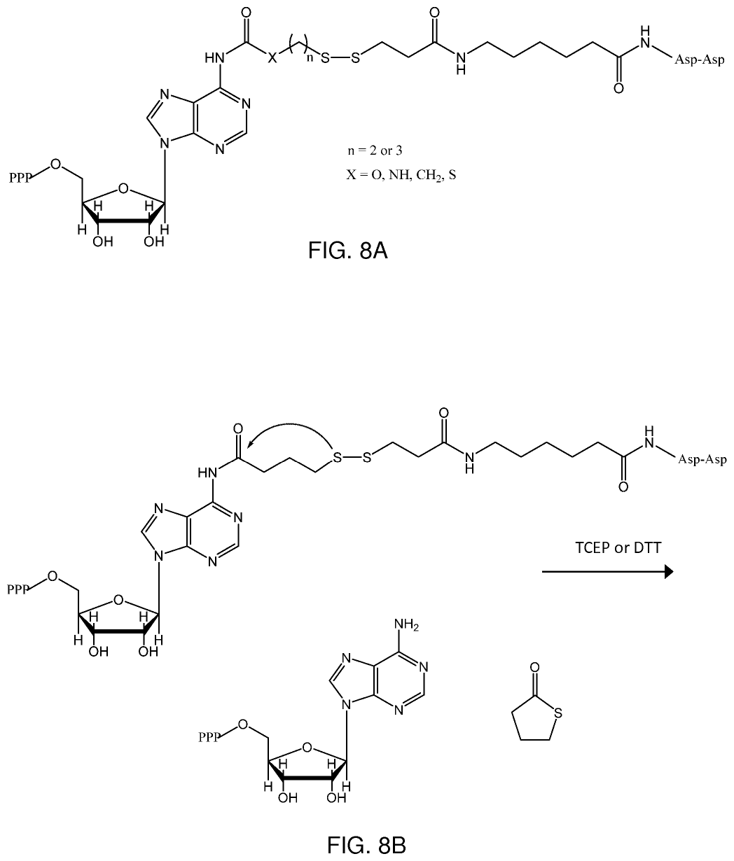

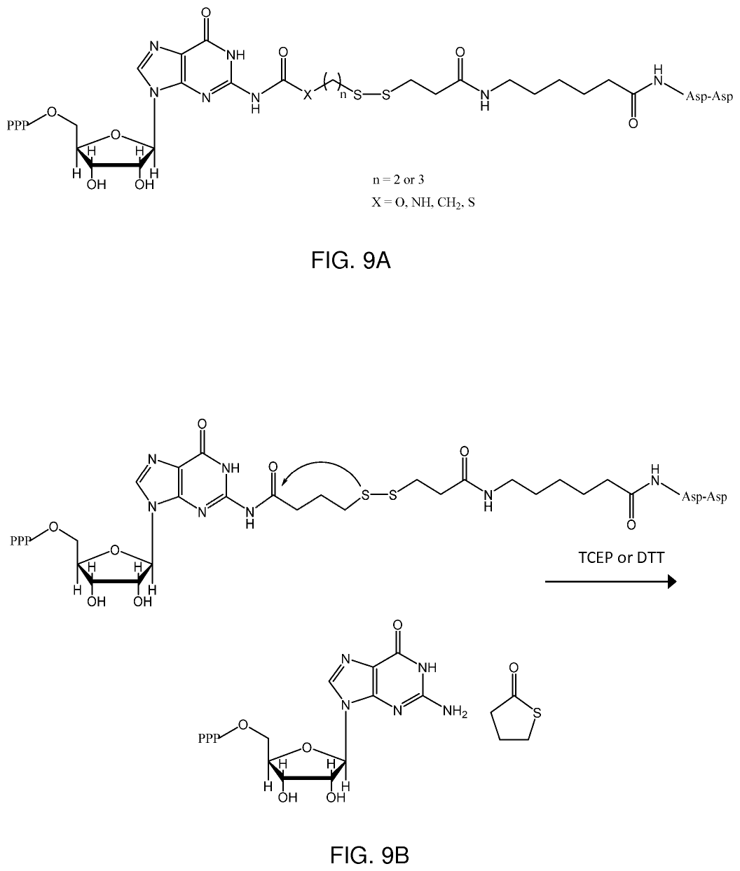

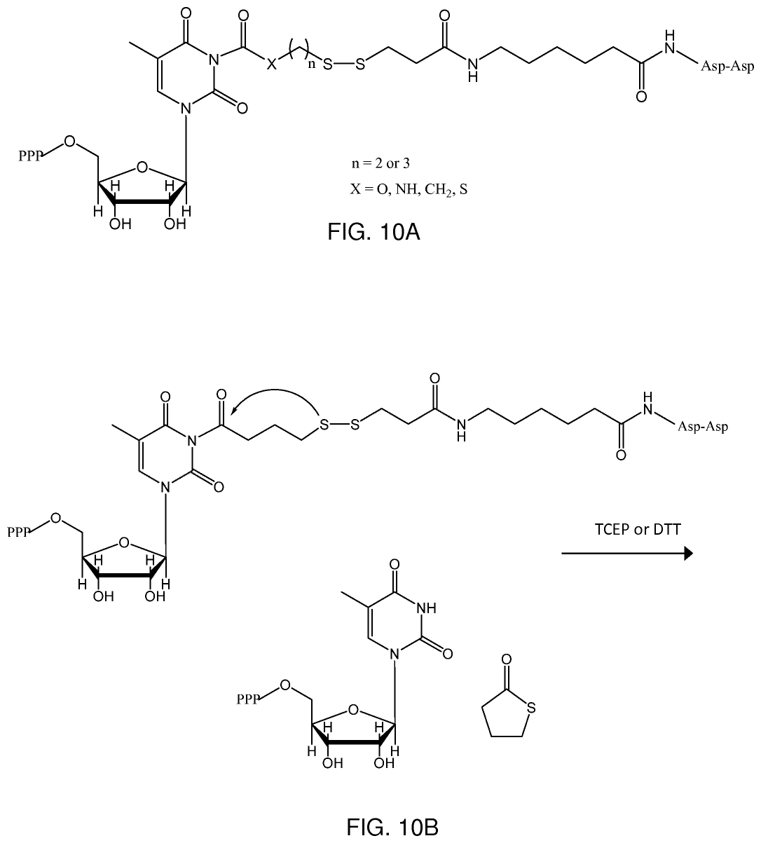

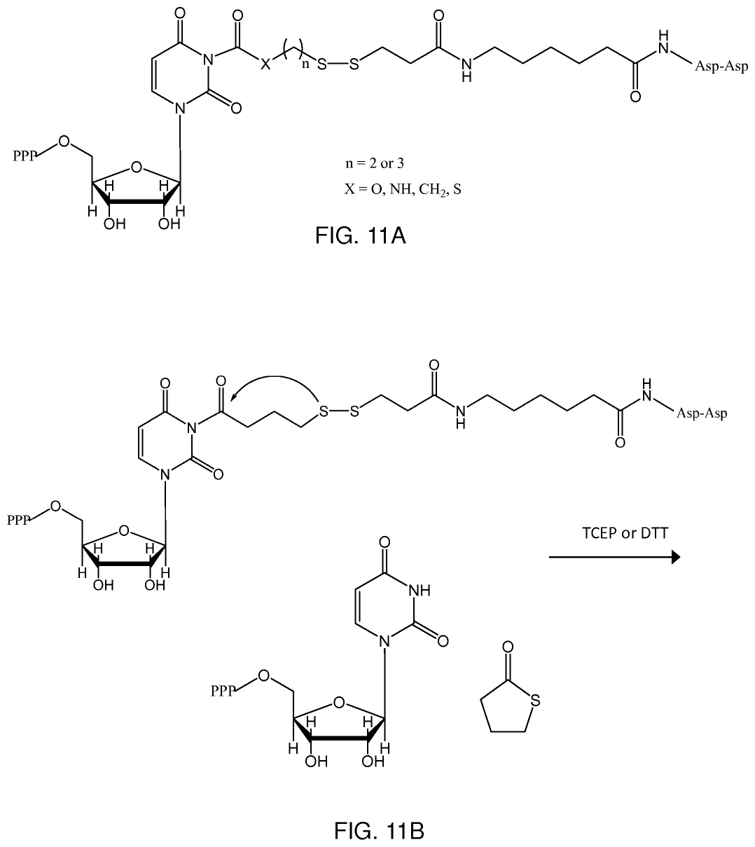

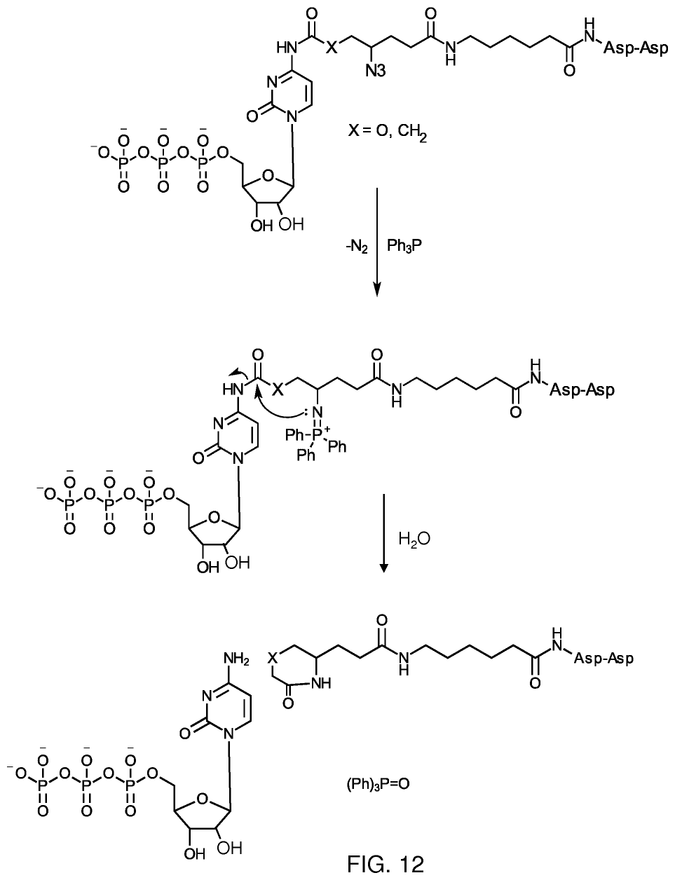

The methodologies of Scheme 1 can be used to produce corresponding ribonucleotide analogs, e.g., as shown in FIGS. 7-10, by starting with the appropriate ribonucleotide reactants. Ribonucleotide analogs comprising the Staudinger linker can also be created using Scheme 2 in order to form the needed ribonucleotide analogs, including, e.g., CTP analogs, as shown in FIG. 12. Furthermore, all of the ribonucleotide analogs, i.e., C, A, T, G, U, can be formed using a reaction similar to Scheme 2.

### Enzymes

The methods of the invention employ nucleotidyl transferases to assemble the nucleotide analogs into polynucleotides. Nucleotidyl transferases include several families of related transferase and polymerase enzymes. Some nucleotidyl transferases polymerize deoxyribonucleotides more efficiently than ribonucleotides, some nucleotidyl transferases polymerize ribonucleotides more efficiently than deoxyribonucleotides, and some nucleotidyl transferases polymerize ribonucleotides and deoxyribonucleotides at approximately the same rate.

Of particular import to the invention, transferases having polymerase activity, such as terminal deoxynucleotidyl transferase (TdT), are capable of catalyzing the addition of deoxyribonucleotides to the 3′ end of a nucleotide chain, thereby increasing chain length in DNA nucleotides. TdT will only catalyze the addition of 1-2 ribonucleotides to the growing end of a DNA strand which could be useful in the construction of site specific DNA-RNA chimeric polynucleotides. In particular, calf thymus TdT, sourced from engineered E. coli, is suitable for use with the invention and available from commercial sources such as Thermo Scientific (Pittsburgh, PA). The amino acid sequence corresponding to calf TdT is listed in Table 1 as SEQ ID NO. 1.

TABLE 1
Amino Acid Sequence of Bovine TdT

SEQ ID NO. 1:
MAQQRQHQRL PMDPLCTASS GPRKKRPRQV GASMASPPHD
IKFQNLVLFI LEKKMGTTRR NFLMELARRK GFRVENELSD
SVTHIVAENN SGSEVLEWLQ VQNIRASSQL ELLDVSWLIE
SMGAGKPVEI TGKHQLVVRT DYSATPNPGF QKTPPLAVKK
ISQYACQRKT TLNNYNHIFT DAFEILAENS EFKENEVSYV
TFMRAASVLK SLPFTIISMK DTEGIPCLGD KVKCIIEEII
EDGESSEVKA VLNDERYQSF KLFTSVFGVG LKTSEKWFRM
GFRSLSKIMS DKTLKFTKMQ KAGFLYYEDL VSCVTRAEAE
AVGVLVKEAV WAFLPDAFVT MTGGFRRGKK IGHDVDFLIT
SPGSAEDEEQ LLPKVINLWE KKGLLLYYDL VESTFEKFKL
PSRQVDTLDH FQKCFLILKL HHQRVDSSKS NQQEGKTWKA
IRVDLVMCPY ENRAFALLGW TGSRQFERDI RRYATHERKM
MLDNHALYDK TKRVFLKAES EEEIFAHLGL DYIEPWERNA

The nucleotide sequence corresponding to calf TdT is listed in Table 2 as SEQ ID NO. 2.

TABLE 2
Nucleic Acid Sequence of Bovine TdT

SEQ ID NO. 2:
ctcttctgga gataccactt gatggcacag cagaggcagc
atcagcgtct tcccatggat ccgctgtgca cagcctcctc
aggccctcgg aagaagagac ccaggcaggt gggtgcctca
atggcctccc ctcctcatga catcaagttt caaaatttgg
tcctcttcat tttggagaag aaaatgggaa ccacccgcag
aaacttcctc atggagctgg ctcgaaggaa aggtttcagg
gttgaaaatg agctcagtga ttctgtcacc cacattgtag
cagaaaacaa ctctggttca gaggttctcg agtggcttca
ggtacagaac ataagagcca gctcgcagct agaactcctt
gatgtctcct ggctgatcga aagtatggga gcaggaaaac
cagtggagat tacaggaaaa caccagcttg ttgtgagaac
agactattca gctaccccaa acccaggctt ccagaagact
ccaccacttg ctgtaaaaaa gatctcccag tacgcgtgtc
aaagaaaaac cactttgaac aactataacc acatattcac
ggatgccttt gagatactgg ctgaaaattc tgagtttaaa
gaaaatgaag tctcttatgt gacatttatg agagcagctt
ctgtacttaa atctctgcca ttcacaatca tcagtatgaa
ggatacagaa ggaattccct gcctggggga caaggtgaag
tgtatcatag aggaaattat tgaagatgga gaaagttctg
aagttaaagc tgtgttaaat gatgaacgat atcagtcctt
caaactcttt acttctgttt ttggagtggg actgaagaca
tctgagaaat ggttcaggat ggggttcaga tctctgagta
aaataatgtc agacaaaacc ctgaaattca caaaaatgca
gaaagcagga tttctctatt atgaagacct tgtcagctgc
gtgaccaggg ccgaagcaga ggcggttggc gtgctggtta
aagaggctgt gtgggcattt ctgccggatg cctttgtcac
catgacagga ggattccgca ggggtaagaa gattgggcat
gatgtagatt ttttaattac cagcccagga tcagcagagg
atgaagagca acttttgcct aaagtgataa acttatggga
aaaaaaggga ttacttttat attatgacct tgtggagtca
acatttgaaa agttcaagtt gccaagcagg caggtggata
ctttagatca ttttcaaaaa tgctttctga ttttaaaatt
gcaccatcag agagtagaca gtagcaagtc caaccagcag
gaaggaaaga cctggaaggc catccgtgtg gacctggtta
tgtgccccta cgagaaccgt gcctttgccc tgctaggctg
gactggctcc cggcagtttg agagagacat ccggcgctat
gccacacacg agcggaagat gatgctggat aaccacgctt
tatatgacaa gaccaagagg gtatttctca aagcggaaag
tgaagaagaa atctttgcac atctgggatt ggactacatt
gaaccatggg aaagaaatgc ttaggagaaa gctgtcaact
tttttctttt ctgttctttt tttcaggtta gacaaattat
gcttcatatt ataatgaaag atgccttagt caagtttggg
attctttaca ttttaccaag atgtagattg cttctagaaa
taagtagttt tggaaacgtg atcaggcacc ccctgggtta
tgctctggca agccatttgc aggactgatg tgtagaactc
gcaatgcatt ttccatagaa acagtgttgg aattggtggc
tcatttccag ggaagttcat caaagcccac tttgcccaca
gtgtagctga aatactgtat acttgccaat aaaaatagga
aac

While commercially available TdT is suitable for use with the methods of the invention, modified TdT, e.g., having an amino acid sequence at least 95% in common with SEQ ID NO. 1, e.g., having an amino acid sequence at least 98% in common with SEQ ID NO. 1, e.g., having an amino acid sequence at least 99% in common with SEQ ID NO. 1, may be used with the methods of the invention. An organism that expresses a suitable nucleotidyl transferase may comprise a nucleic acid sequence at least 95% in common with SEQ ID NO. 2, e.g., at least 98% in common with SEQ ID NO. 2, e.g., at least 99% in common with SEQ ID NO. 2. In some instances, a modified TdT will result in more efficient generation of polynucleotides or allow better control of chain length. Other modifications to the TdT may change the release characteristics of the enzyme, thereby reducing the need for aqueous reducing agents such as TCEP or DTT.

For the synthesis of RNA polynucleotides, a nucleotidyl transferase like E. coli poly (A) polymerase can be used to catalyze the addition of ribonucleotides to the 3′ end of a ribonucleotide initiator. In other embodiments, E. coli poly (U) polymerase may be more suitable for use with the methods of the invention. Both E. coli poly (A) polymerase and E. coli poly (U) polymerase are available from New England Biolabs (Ipswich, MA). The amino acid and nucleotide sequences for E. coli Poly (A) polymerase and E. coli Poly (U) polymerase are reproduced below. Modified E. coli Poly (A) polymerase or E. coli Poly (U) polymerase may be suitable for use with the methods of the invention. For example, an enzyme, having an amino acid sequence at least 95% in common with SEQ ID NO. 3, e.g., having an amino acid sequence at least 98% in common with SEQ ID NO. 3, e.g., having an amino acid sequence at least 99% in common with SEQ ID NO. 3, may be used with the methods of the invention. An organism that expresses a suitable enzyme may comprise a nucleic acid sequence at least 95% in common with SEQ ID NO. 4, e.g., at least 98% in common with SEQ ID NO. 4, e.g., at least 99% in common with SEQ ID NO. 4. Alternatively, an enzyme having an amino acid sequence at least 95% in common with SEQ ID NO. 5, e.g., having an amino acid sequence at least 98% in common with SEQ ID NO. 5, e.g., having an amino acid sequence at least 99% in common with SEQ ID NO. 5, may be used with the methods of the invention. An organism that expresses a suitable enzyme may comprise a nucleic acid sequence at least 95% in common with SEQ ID NO. 6, e.g., at least 98% in common with SEQ ID NO. 6, e.g., at least 99% in common with SEQ ID NO. 6.

TABLE 3
Amino Acid Sequence of E. coli Poly(A)
polymerase

SEQ ID NO. 3:
MFTRVANFCR KVLSREESEA EQAVARPQVT VIPREQHAIS
RKDISENALK VMYRLNKAGY EAWLVGGGVR DLLLGKKPKD
FDVTTNATPE QVRKLFRNCR LVGRRFRLAH VMFGPEIIEV
ATFRGHHEGN VSDRTTSQRG QNGMLLRDNI FGSIEEDAQR
RDFTINSLYY SVADFTVRDY VGGMKDLKDG VIRLIGNPET
RYREDPVRML RAVRFAAKLG MRISPETAEP IPRLATLLND
IPPARLFEES LKLLQAGYGY ETYKLLCEYH LFQPLFPTIT
RYFTENGDSP MERIIEQVLK NTDTRIHNDM RVNPAFLFAA
MFWYPLLETA QKIAQESGLT YHDAFALAMN DVLDEACRSL
AIPKRLTTLT RDIWQLQLRM SRRQGKRAWK LLEHPKFRAA
YDLLALRAEV ERNAELQRLV KWWGEFQVSA PPDQKGMLNE
LDEEPSPRRR TRRPRKRAPR REGTA

The nucleotide sequence corresponding to E. coli poly (A) polymerase is listed in Table 4 as SEQ ID NO. 4.

TABLE 4
Nucleotide Sequence of E. coli Poly(A)
polymerase

SEQ ID NO. 4:
atttttaccc gagtcgctaa tttttgccgc aaggtgctaa
gccgcgagga aagcgaggct gaacaggcag tcgcccgtcc
acaggtgacg gtgatcccgc gtgagcagca tgctatttcc
cgcaaagata tcagtgaaaa tgccctgaag gtaatgtaca
ggctcaataa agcgggatac gaagcctggc tggttggcgg
cggcgtgcgc gacctgttac ttggcaaaaa gccgaaagat
tttgacgtaa ccactaacgc cacgcctgag caggtgcgca
aactgttccg taactgccgc ctggtgggtc gccgtttccg
tctggctcat gtaatgtttg gcccggagat tatcgaagtt
gcgaccttcc gtggacacca cgaaggtaac gtcagcgacc
gcacgacctc ccaacgcggg caaaacggca tgttgctgcg
cgacaacatt ttcggctcca tcgaagaaga cgcccagcgc
cgcgatttca ctatcaacag cctgtattac agcgtagcgg
attttaccgt ccgtgattac gttggcggca tgaaggatct
gaaggacggc gttatccgtc tgattggtaa cccggaaacg
cgctaccgtg aagatccggt acgtatgctg cgcgcggtac
gttttgccgc caaattgggt atgcgcatca gcccggaaac
cgcagaaccg atccctcgcc tcgctaccct gctgaacgat
atcccaccgg cacgcctgtt tgaagaatcg cttaaactgc
tacaagcggg ctacggttac gaaacctata agctgttgtg
tgaatatcat ctgttccagc cgctgttccc gaccattacc
cgctacttca cggaaaatgg cgacagcccg atggagcgga
tcattgaaca ggtgctgaag aataccgata cgcgtatcca
taacgatatg cgcgtgaacc cggcgttcct gtttgccgcc
atgttctggt acccactgct ggagacggca cagaagatcg
cccaggaaag cggcctgacc tatcacgacg ctttcgcgct
ggcgatgaac gacgtgctgg acgaagcctg ccgttcactg
gcaatcccga aacgtctgac gacattaacc cgcgatatct
ggcagttgca gttgcgtatg tcccgtcgtc agggtaaacg
cgcatggaaa ctgctggagc atcctaagtt ccgtgcggct
tatgacctgt tggccttgcg agctgaagtt gagcgtaacg
ctgaactgca gcgtctggtg aaatggtggg gtgagttcca
ggtttccgcg ccaccagacc aaaaagggat gctcaacgag
ctggatgaag aaccgtcacc gcgtcgtcgt actcgtcgtc
cacgcaaacg cgcaccacgt cgtgagggta ccgcatga

TABLE 5
Amino Acid Sequence of E. coli Poly(U)
polymerase

SEQ ID NO. 5:
GSHMSYQKVP NSHKEFTKFC YEVYNEIKIS DKEFKEKRAA
LDTLRLCLKR ISPDAELVAF GSLESGLALK NSDMDLCVLM
DSRVQSDTIA LQFYEELIAE GFEGKFLQRA RIPIIKLTSD
TKNGFGASFQ CDIGFNNRLA IHNTLLLSSY TKLDARLKPM
VLLVKHWAKR KQINSPYFGT LSSYGYVLMV LYYLIHVIKP
PVFPNLLLSP LKQEKIVDGF DVGFDDKLED IPPSQNYSSL
GSLLHGFFRF YAYKFEPREK VVTFRRPDGY LTKQEKGWTS
ATEHTGSADQ IIKDRYILAI EDPFEISHNV GRTVSSSGLY
RIRGEFMAAS RLLNSRSYPI PYDSLFEEA

The nucleotide sequence corresponding to E. coli poly (U) polymerase is listed in Table 6 as SEQ ID NO. 6.

TABLE 6
Nucleotide Sequence of E. coli Poly(A)
polymerase

SEQ ID NO. 6:
ggcagccata tgagctatca gaaagtgccg aacagccata
aagaatttac caaattttgc tatgaagtgt ataacgaaat
taaaattagc gataaagaat ttaaagaaaa acgcgcggcg
ctggataccc tgcgcctgtg cctgaaacgc attagcccgg
atgcggaact ggtggcgttt ggcagcctgg aaagcggcct
ggcgctgaaa aacagcgata tggatctgtg cgtgctgatg
gatagccgcg tgcagagcga taccattgcg ctgcagtttt
atgaagaact gattgcggaa ggctttgaag gcaaatttct
gcagcgcgcg cgcattccga ttattaaact gaccagcgat
accaaaaacg gctttggcgc gagctttcag tgcgatattg
gctttaacaa ccgcctggcg attcataaca ccctgctgct
gagcagctat accaaactgg atgcgcgcct gaaaccgatg
gtgctgctgg tgaaacattg ggcgaaacgc aaacagatta
acagcccgta ttttggcacc ctgagcagct atggctatgt
gctgatggtg ctgtattatc tgattcatgt gattaaaccg
ccggtgtttc cgaacctgct gctgagcccg ctgaaacagg
aaaaaattgt ggatggcttt gatgtgggct ttgatgataa
actggaagat attccgccga gccagaacta tagcagcctg
ggcagcctgc tgcatggctt ttttcgcttt tatgcgtata
aatttgaacc gcgcgaaaaa gtggtgacct ttcgccgccc
ggatggctat ctgaccaaac aggaaaaagg ctggaccagc
gcgaccgaac ataccggcag cgcggatcag attattaaag
atcgctatat tctggcgatt gaagatccgt ttgaaattag
ccataacgtg ggccgcaccg tgagcagcag cggcctgtat
cgcattcgcg gcgaatttat ggcggcgagc cgcctgctga
acagccgcag ctatccgatt ccgtatgata gcctgtttga
agaagcg

As discussed above, the inhibitor coupled to the nucleotide analog will cause the transferase, e.g., TdT, to not release from the polynucleotide or prevent other analogs from being incorporated into the growing chain. A charged moiety results in better inhibition, however, research suggests that the specific chemical nature of the inhibitor is not particularly important. For example, both phosphates and acidic peptides can be used to inhibit enzymatic activity. See, e.g., Bowers et al., Nature Methods, vol. 6, (2009) p. 593-95, and U.S. Pat. No. 8,071,755, both of which are incorporated herein by reference in their entireties. In some embodiments, the inhibitor will include single amino acids or dipeptides, like -(Asp) 2, however the size and charge on the moiety can be adjusted, as needed, based upon experimentally determined rates of first nucleotide incorporation and second nucleotide incorporation. That is, other embodiments may use more or different charged amino acids or other biocompatible charged molecule.

Other methods of nucleotide synthesis may be used to build de novo oligonucleotides in a template independent fashion using nucleotidyl transferases or modified nucleotidyl transferases. In one embodiment, the polymerase/transferase enzymes can be modified so that they cease nucleotide addition when they encounter a modification to the phosphate of a 3′-unmodified dNTP analog. This scheme would require a deblocking reagent/reaction that modifies the phosphate end of the nucleotide analog, which frees up the nascent strand for subsequent nucleotide incorporation. Preferred embodiments of this approach would use nucleotide analogs modified only at the phosphates (alpha, beta or gamma) although modifications of the purine/pyrimidine base of the nucleotide are allowed.

Another embodiment for using non-template dependent polymerase/transferase enzymes would be to using protein engineering or protein evolution to modify the enzyme to remain tightly bound and inactive to the nascent strand after each single nucleotide incorporation, thus preventing any subsequent incorporation until such time as the polymerase/transferase is released from the strand by use of a releasing reagent/condition. Such modifications would be selected to allow the use of natural unmodified dNTPs instead of reversible terminator dNTPs. Releasing reagents could be high salt buffers, denaturants, etc. Releasing conditions could be high temperature, agitation, etc. For instance, mutations to the Loop1 and SD1 regions of TdT have been shown to dramatically alter the activity from a template-independent activity to more of a template dependent activity. Specific mutations of interest include but are not limited to 3384/391/392, del loop1 (386 398), D339A, F401A, and Q402K403C404→E402R403S404. Other means of accomplishing the goal of a post-incorporation tight binding TdT enzyme could include mutations to the residues responsible for binding the three phosphates of the initiator strand including but not limited to K261, R432, and R454.

Another embodiment for using non-template dependent polymerase/transferase enzymes would be to use protein engineering or protein evolution to modify the enzyme to accept 3-blocked reversible terminators with high efficiency. Most naturally occurring polymerase/transferase enzymes will not incorporate 3′-blocked reversible terminators due to steric constraints in the active site of the enzyme. Modifying either single or several aa residues in the active site of the enzyme can allow the highly efficient incorporation of 3′-blocked reversible terminators into a support bound initiator in a process completely analogous to that described above. After incorporation, the 3′-reversible terminator is removed with a deblocking reagent/condition thus generating a completely natural (scarless) single strand molecule ready for subsequent controlled extension reactions. There are few residues close to the 3′-OH of the incoming dNTP which explains the propensity of TdT for incorporating ribonucleotide triphosphates as readily as deoxyribonucleotide triphosphates; residues including but not limited to those between 1 and 2 especially R334, Loop1, and those between a13 and a14, especially R454, are likely targets for mutagenesis to accommodate the bulk of 3′-reversible terminator groups and allow their efficient incorporation. Another embodiment for using template-dependent polymerases would be to use the either 3′blocked or 3′unblocked dNTP analogs with a plurality of primer-template pairs attached to a solid support.

Another embodiment for using non-template dependent polymerase/transferase enzymes can use protein engineering or protein evolution to modify the enzyme to optimize the use of each of the four different nucleotides or even different modified nucleotide analogs in an analog specific manner. Nucleotide specific or nucleotide analog specific enzyme variants could be engineered to possess desirable biochemical attributes like reduced Km or enhanced addition rate which would further reduce the cost of the synthesis of desired polynucleotides.

### Solid State Synthesis

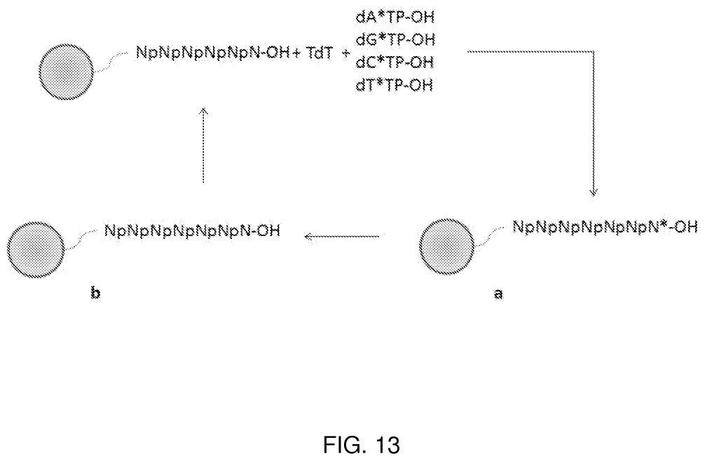

The methods of the invention can be practiced under a variety of reaction conditions, however the orderly construction and recovery of desired polynucleotides will, in most cases, require a solid support to which the polynucleotides can be grown. In some embodiments, the methods include the enzymatically mediated synthesis of polynucleotides on a solid support, as illustrated in FIG. 7. When used in conjunction with the cleavable terminator nucleotide triphosphate (NTP) analogs discussed above, it is possible to construct specific polynucleotide sequences of DNA as well as RNA by using, for example, TdT or poly (A) polymerase in an aqueous environment. As shown in FIG. 13, the TdT can be used to effect the stepwise construction of custom polynucleotides by extending the polynucleotide sequence a stepwise fashion. As discussed previously, the inhibitor group of each NTP analog causes the enzyme to stop with the addition of a nucleotide. After each nucleotide extension step, the reactants are washed away from the solid support prior to the removal of the inhibitor by cleaving the linker, and then new reactants are added, allowing the cycle to start anew. At the conclusion of n cycles of extension-remove-deblocking-wash, the finished full-length, single-strand polynucleotide is complete and can be cleaved from the solid support and recovered for subsequent use in applications such as DNA sequencing or PCR. Alternatively, the finished, full-length, single-strand polynucleotide can remain attached to the solid support for subsequent use in applications such as hybridization analysis, protein or DNA affinity capture. In other embodiments, partially double-stranded DNA can be used as an initiator, resulting in the synthesis of double-stranded polynucleotides.

Solid supports suitable for use with the methods of the invention may include glass and silica supports, including beads, slides, pegs, or wells. In some embodiments, the support may be tethered to another structure, such as a polymer well plate or pipette tip. In some embodiments, the solid support may have additional magnetic properties, thus allowing the support to be manipulated or removed from a location using magnets. In other embodiments, the solid support may be a silica coated polymer, thereby allowing the formation of a variety of structural shapes that lend themselves to automated processing.

### Synthesizers

To capitalize on the efficiency of the disclosed methods, an aqueous phase DNA synthesizer can be constructed to produce desired polynucleotides in substantial quantities. In one embodiment, a synthesizer will include four wells of the described NTP analog reagents, i.e., dCTP, dATP, dGTP, and dTTP, as well as TdT at concentrations sufficient to effect polynucleotide growth. A plurality of initiating sequences can be attached to a solid support that is designed to be repeatedly dipped into each of the four wells, e.g., using a laboratory robot. The robot could be additionally programmed to rinse the solid support in wash buffer between nucleotide additions, cleave the linking group by exposing the support to a deblocking agent, and wash the solid support a second time prior to moving the solid support to the well of the next desired nucleotide. With simple programming, it is possible to create useful amounts of desired nucleotide sequences in a matter of hours, and with substantial reductions hazardous waste. Ongoing synthesis under carefully controlled conditions will allow the synthesis of polynucleotides with lengths in the thousands of base pairs. Upon completion, the extension products are released from the solid support, whereupon they can be used as finished nucleotide sequences.

A highly parallel embodiment could consist of a series of initiator-solid supports on pegs in either 96 or 384 well formats that could be individually retracted or lowered so that the pegs can be indexed to contact the liquids in the wells in a controlled fashion. The synthesizer could thus consist of the randomly addressable peg device, four enzyme-dNTP analog reservoirs in the same format as the peg device (96 or 384 spacing), additional reagent reservoirs (washing, deblocking, etc.) in the same format as the peg device (96 or 384 spacing), and a transport mechanism (e.g., a laboratory robot) for moving the peg device from one reservoir to another in a user programmable controlled but random access fashion. Care must be taken to avoid contaminating each of the four enzyme-dNTP reservoirs since the contents are reused throughout the entire synthesis process to reduce the cost of each polynucleotide synthesis.

In alternative embodiments, the reagents (e.g., nucleotide analogs, enzymes, buffers) will be moved between solid supports, allowing the reagents to be recycled. For example, a system of reservoirs and pumps can move four different nucleotide analog solutions, wash buffers, and/or reducing agent solutions between one or more reactors in which the oligonucleotides will be formed. The reactors and pumps can be conventional, or the devices may be constructed using microfluidics. Because of the non-anhydrous (aqueous) nature of the process, no special care needs to be taken in the design of the hardware used to eliminate exposure to water. The synthesis process can take place with only precautions to control evaporative loss. A highly parallel embodiment could consist of a monolithic series of initiator-solid supports on pegs in either 96 or 384 well format that can be interfaced to a series of wells in the same matching format. Each well would actually be a reaction chamber that is fed by four enzyme-dNTP analog reservoirs and additional reagent reservoirs (washing, deblocking, etc.) with appropriate valves. Provisions would be made in the fluidics logic to recover the enzyme-dNTP reactants in a pristine fashion after each extension reaction since they are reused throughout the entire synthesis process to reduce the cost of each polynucleotide synthesis. In other embodiments, a system of pipetting tips could be used to add and remove reagents.

After synthesis, the released extension products can be analyzed by high resolution PAGE to determine if the initiators have been extended by the anticipated number of bases compared to controls. A portion of the recovered synthetic DNA may also be sequenced to determine if the synthesized polynucleotides are of the anticipated sequence.

Because the synthesizers are relatively simple and do not require the toxic components needed for phosphoramidite synthesis, synthesizers of the invention will be widely accessible for research institutions, biotechs, and hospitals. Additionally, the ability to reuse/recycle reagents will reduce the waste produced and help reduce the costs of consumables. The inventors anticipate that the methods and systems will be useful in a number of applications, such as DNA sequencing, PCR, and synthetic biology.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for non-template dependent oligonucleotide synthesis, the method comprising:
exposing a nucleic acid strand to a terminal transferase enzyme capable of incorporating a single nucleotide and remaining bound to the strand and preventing further nucleotide incorporation until exposed to a releasing agent or releasing condition.

2. The method of claim 1, wherein said single nucleotide is a nucleotide analog.

3. The method of claim 1, wherein the terminal transferase enzyme is a modified terminal deoxynucleotidyl transferase (TdT) enzyme.

4. The method of claim 3, wherein the modification comprises a mutation allowing the covalent attachment of a nucleotide analog to the TdT enzyme.

5. The method of claim 1, wherein the releasing reagent comprises a salt buffer or a denaturant or a reducing agent or elevated pH.

6. The method of claim 1, wherein the releasing condition is a temperature increase or agitation.

7. A method for non-template dependent oligonucleotide synthesis, the method comprising:
exposing a covalent blocker-nucleic acid strand complex to an exonuclease to remove un complexed, unmodified nucleic acid strands;
removing the exonuclease; and
exposing the covalent blocker-nucleic acid strand complex to a releasing agent or a releasing condition.
